(19) United States
(12) Patent Application Publication
(10) Pub. No.: US 2005/0163776 A1
(43) Pub. Date: Jul. 28, 2005

(54) Treatment of tse infection

(76) Inventors: Neil David Hammond Raven, Wiltshire (GB); John Mark Sutton, Wiltshire (GB); Heather Murdoch, Wiltshire (GB)

Correspondence Address: Evan Law Group, Suite 350, 566 West Adams, Chicago, IL, 60661, US

(21) Appl. No.: 10/508,296

(22) PCT Filed: Mar. 20, 2003
(86) PCT No.: PCT/GB03/01295

(30) Foreign Application Priority Data
Mar. 20, 2002 (GB) 0206584.5
Jul. 11, 2002 (GB) 0216098.4

(51) Int. Cl. A61K 39/395

(52) U.S. Cl. 424/145.1

(57) ABSTRACT

Transmissible spongiform encephalopathy (TSE) infection is treated by administration of an antibody that binds to prion dimer. A conjugate of a carrier and a fragment of a prion protein, optionally in oligomeric form and optionally having cyclic regions, is used to stimulate antibody production.

The invention relates to methods and compositions for the treatment of infection by transmissible spongiform encephalopathy (TSE) agents.

Transmissible spongiform encephalopathies (TSEs) are a group of fatal neurological diseases that include Creutzfeld-Jacob disease (CJD) and Kuru in humans, bovine spongiform encephalopathy (BSE) in cattle and Scrapie in sheep. TSEs are characterised by the conversion of a normal host protein into a pathogenic protein within the brain tissue of an infected animal. The pathogenic form of the protein is often referred to as a prion and is highly resistant to physical and chemical degradation. The prion is believed to be the transmissive agent through which the TSE disease is passed on between animals.

There has been considerable public alarm in recent years over the risks associated with consumption of meat products, and especially beef, potentially infected with BSE, the bovine form of TSE. Much of this concern is associated with the belief that the BSE prion when eaten by a human may in some cases cause the incurable human form of the disease, referred to as variant CJD (vCJD).

For those infected with TSE the prognosis is poor, with no effective therapies being available. Often the time from diagnosis to death is short and uncomfortable both for the patient and for those around and caring for the patient.

It is known to raise antibodies to fragments of prion proteins. Souan et al, Eur. J. Immunol. 2001: 31, pp 2338-2346 describe peptides that induce both T- and B-cell responses. However, no reduction in prion protein tumour was achieved using the antibodies obtained.

A difficulty in raising antibodies to prion proteins is that peptide fragments are poor immunogens, and the antibodies obtained are frequently of poor affinity. If pure disease-causing prion protein itself is used this has the consequence that the resultant composition is likely to contain disease-causing protein, and hence be unusable in the clinic.

An anti-prion antibody, mAb 6H4 is also available commercially from Prionics, Switzerland. This antibody can be used to detect prion protein, typically using a second antibody conjugated to a detectable marker, which second antibody binds to the first.

A difficulty that has been discovered by the present inventors is that binding of this antibody to equipment suspected of being contaminated with prion, or equipment that is suspected to be contaminated but which has been subjected to treatment intended to destroy the prion, does not correlate with infectivity. It has, for instance, been discovered by the inventors that prion-infected mouse brain homogenate, digested with protease, run on SDS-PAGE, then probed with anti-prion antibody, shows a negative result, that is to say absence of antibody binding. This material nevertheless retains infectivity.

An object of the invention is to provide effective therapy, curative and/or preventative, for those suffering from or at risk of infection or other disease caused by TSE agents.

A further object is to provide alternative and, in specific embodiments, improved production of antibodies that bind to prion proteins and can be used to treat prion disease.

Accordingly, the invention provides a method of treatment of TSE infection, comprising administering an antibody that binds to a dimer of a prion protein.

The antibody is preferably specific to the dimer, that is to say it binds to the dimer but substantially does not bind to the monomer form of the prion, and may be obtained by methods described in detail below.

A further aspect of the invention provides a pharmaceutical composition, for treatment of TSE infection, comprising an antibody that binds to a dimer of a prion protein.

An antibody of the invention is suitably obtained by immunising an animal with an antigen that comprises prion protein or an analogue thereof, or a fragment of the protein or analogue, obtaining an extract therefrom which contains antibodies, and isolating from said extract antibodies which bind prion dimer. Mouse, sheep, human and bovine (as well as other) prion protein sequences are known and hence it is straightforward to prepare a peptide that is a fragment of, say, at least 7, preferably at least 10, more preferably at least 14 amino acids. An analogue can be prepared by comparison of respective prion protein sequences and synthesizing a composite with regions derived from one or more prion protein source. Alternatively a synthetic sequence is prepared in which up to 2 amino acids in every 10 are substituted.

The antigen of the invention may comprise a mixture of peptides. These peptides may comprise different regions from one or more prion protein sequences, and/or the same regions from one or more prion protein sequences, which same regions may contain intraspecies variation.

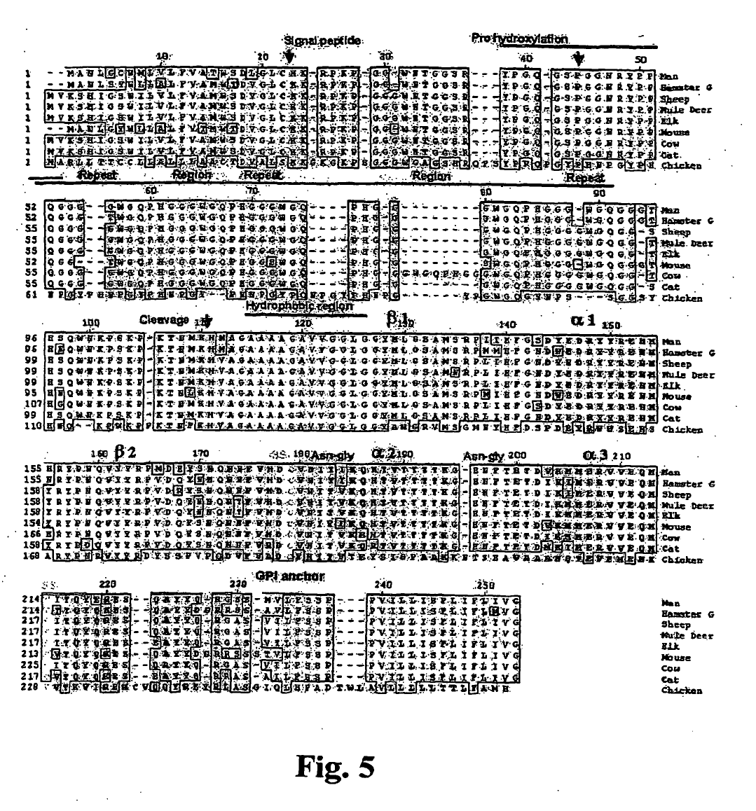

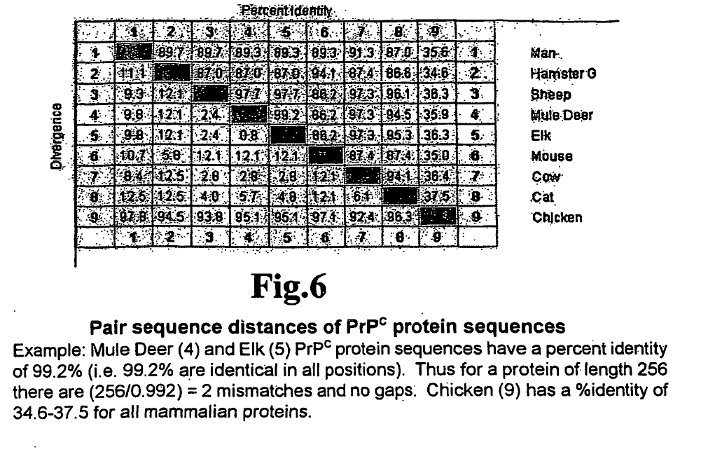

It is known that there is a high degree of homology between the prion protein sequences of many mammals—both important functional residues and structural elements are highly conserved. Accordingly, it is possible to use these conserved sequence regions, or fragments thereof, to prepare the immunising peptides of the present invention. These peptides can comprise a variety of different regions from one or more prion protein sequences. The high degree of prion protein sequence homology between species is set out in FIGS. 5 and 6.

In FIG. 5, the human prion protein precursor PrP^{c} was aligned with Hamster, Sheep, Mule Deer, Elk, Mouse, Cow, Cat and Chicken homologous proteins using the ClustalW multiple alignment program (Higgins et al, 1994). The sequences used for this alignment were taken from the SWISSPROT database and the database accession numbers of the sequences used are as follows:—

TABLE 1
SWISSPROT database accession numbers.

| Species | |
|---|---|
| Name | SWISSPORT accession No. |
| Human | P04156 |
| Mouse | P04295 |
| Hamster, | P04273 |
| Golden | |
| Sheep | P23907 |
| Cow | P10279 |
| Elk | P79142 |
| Mule Deer | P47852 |
| Cat | O18754 |
| Chicken | P27177 (representative of |
| | avian species) |

Important structural features are marked above the aligned sequences of FIG. 5 and numbering refers to the Human PrP^{c} sequence. Some of the important structural features of the sequences are as follows (terms in brackets indicate how these features are marked on FIG. 5):—
1. The signal peptide is cleaved at position 22/23 to yield the mature protein (Signal peptide)
2. The N-terminus region is largely unstructured and flexible but residues 37-53 can form an extended PPII helix forming a hydroxylation site at Pro44 (Pro hydroxylation)
3. The N-terminus also comprises a segment of 5/6 repeats which are implicated in Copper binding (Repeat Region)
4. Proteolysis can occur between Lys110 and His 111 (Cleavage)
5. There is a short hydrophobic residue region (Hydrophobic region) believed to be a transmembrane segment.
6. The C-terminus is characterised by a bundle of three α-helices (a 1-3) and two β-sheets (b1-2)
7. A di-sulphide bridge occurs between residues Cys179 and Cys214 (-SS-)
8. Asparagine residues Asn181 and Asn197 are available for N-glycosylation (Carbohydrate groups; the conserved motif being Asn-X-Thr (Asn-Gly).
9. Residue Ser231 provides the residue where the GPI (glycolipid) anchor moiety is attached (GPI anchor).

On inspection of the multiple alignment of FIG. 5, the following functionally important residues were absolutely conserved in the mammalian species shown:
Signal Peptide cleavage site (C23-K24)
Pro44 hydroxylation site,
Proteolytic cleavage site (KI 10-H 111)
Arg-glycosylation sites (R181, R197)
Disulphide bridge (C179, C214)
GPI anchor (S231)

Regions which differ tend to be in the N-terminal sequence and some of the structural elements (e.g. α-helices), although the β-sheets and the hydrophobic region are totally conserved.

FIG. 6 shows the percent identifies derived from the alignment of individual pairs of protein sequences. Again, this figure demonstrates the high level of identity between mammalian species of the prion protein amino acid sequence. Preferably, peptides for use in methods and compositions of the invention are prepared from fragments of the identified conserved regions.

One way to make an antibody selective for prion dimer is to immunise an animal and extract serum. This is run on a prion dimer column to identify antibodies that bind the dimer. These antibodies are then tested for cross-reactivity with prion monomer, with cross-reacting antibodies removed. The removal can be effected using a column loaded with prion monomer, the antibodies that emerge therefrom then being tested for absence of cross-reactivity.

Isolated prion dimer forms a further embodiment of the invention. This isolated material can be obtained from the column in the method described immediately above or simply by cutting out a portion of the SDS-PAGE gel used to resolve prion dimers. Alternatively, other separation techniques can be used to extract the prion dimer from homogenised prion-infected mouse brain. Antibodies that bind to the prion monomer and which are cross reactive can be used to confirm that the material thus obtained is prion dimer and not other protein of the same molecular weight.

In a preferred embodiment of the invention, the antibody is obtained by immunising an animal with a peptide that is or comprises a fragment of prion protein optionally supplemented by a cysteine residue at one or both ends, in specific examples one selected from SEQ ID NO:s 1 to 8. The use of cysteine residues (reference to the sequence or fragment is intended to include reference to the sequence or fragment with 0, 1 or 2 cysteine residues at its ends) offers the advantage that the peptides may form oligomers due to cysteine-cysteine bonds. As a result the immunizing peptides include linear monomers, linear dimers, cyclic monomers, cyclic dimers and other oligomeric forms with repeated prion peptide sequences and cyclic regions. These peptides allow a wider range of immunising antigens to be presented to the animal. Cyclic forms are presented which may more closely mimic the natural form of the disease-forming agent. Dimeric forms, some of them cyclic, are presented which again may more closely mimic the dimeric forms of the disease-causing agent. The invention hence extends to all fragments of all prion sequences described in FIG. 5, wherein the fragments are at least 7 amino acids in length and have cysteine residues at one or both ends.

The peptide may be introduced into the animal via various routes of administration. In a preferred embodiment, the peptide is injected into the peritoneum, a site which is (a) surgically easily accessible, (b) allows a large volume of liquid to be injected at any one time, and (c) allows rapid absorption of the peptide into the bloodstream.

In use, the antibody can be delivered to the systemic circulation. Binding of the antibody to prion dimer results in removal and destruction of that dimer and reduction of infection. It is believed that prion dimer originating in the brain will diffuse across the blood brain barrier and that the presence of antibodies on the systemic side of the barrier will create a concentration gradient as dimer is mopped up after crossing the barrier, this helping to reduce infection and disease.

It is known to conjugate antibodies to transport vectors so that antibodies can cross the blood brain barrier. For example, an antibody can be coupled via avidin/biotin to the OX 26 monoclonal antibody, which latter antibody acts as a transport vector for crossing the blood brain barrier. Further details are described in Bickel et al, Advanced Drug Discovery Reviews 46 (2001) 247-279. A further embodiment of the invention hence lies in an antibody adapted, for example using the technology reviewed by Bickel et al, to cross the blood brain barrier.

A further preferred embodiment of the invention lies in enhancing the immune response to the antigen by use of a carrier. Hence, antibodies are obtained by immunising an animal with an antigen, wherein the antigen comprises a peptide of the invention that is, or comprises a fragment of, a prion protein or an analogue of a prion protein, and wherein the antigen further comprises a carrier covalently linked to the peptide, optionally via a linker. This has the advantage of improved stimulation of antibody production. The carrier can be selected from a wide range of immunogenic carrier substances, e.g. proteins, heat shock proteins, toxoids, including such examples as bacterial proteins and bacterial toxoids, particularly mycobacterial proteins, pertussis proteins and toxoids, diphtheria proteins and toxoids. De Silva et al., Bioconjug Chem 1999 May-June; 10(3); 496-501 describe use of PPD as an immunogenic carrier, and heat shock proteins as carriers are described in Lussow et al, Immunol 1991 October: 21(10); 2297-2302. The animal can be a source of antibodies, in which case antibodies can be obtained from that animal and those that bind to prion dimer identified. The animal can be one being or to be treated. The invention hence also provides methods of treating TSE and/or immunizing an animal against TSE infection, comprising administering the peptide of the invention and/or the antigen of the invention.

It is, in addition, preferred to sensitise the animal to the carrier, either prior to or at the same time as administering the antigen/carrier. Thus, in a particular method described in the examples, antibody production comprises administrating a priming antigen that stimulates an immune response to the carrier. Again, an advantage is improved antibody production. The priming antigen may be carrier on its own or a fragment of the carrier.

In a specific example, the carrier is a Mycobacterial protein and the priming antigen is administered by administering BCG vaccine. Further details of this approach are described for example in Lussow et al, Immunol 1991 October: 21(10); 2297-2302.

Still further aspects of the invention lie in isolated peptides and antigens of the invention and their uses.

The term transmissible spongiform encephalopathy (TSE) agent is intended to encompass all neurological diseases that are apparently transmitted via a pathogenic prion protein intermediate. Such TSEs typically include the human diseases Creutzfeld-Jacob disease (CJD), variant Creutzfeld-Jacob disease (vCJD), Kuru, fatal familial insomnia and Gerstmann-Straussler-Scheinker syndrome. Non-human TSEs include bovine spongiform encephalopathy (BSE), scrapie, feline spongiform encephalopathy, chronic wasting disease, and transmissible mink encephalopathy. Given that vCJD is currently understood to be a human form of BSE, it is apparent that certain TSE agents are capable of crossing the species barrier and that novel TSEs from non-bovine sources could become evident in future. Reference to TSE infection refers also to prion disease.

The antibodies and/or peptides of the invention can be used in an effective therapy, curative and/or preventative, for clearing all or part of the UK herd of scrapie (sheep). In sheep, there are often flocks where up to 20% of the flock catches scrapie. It would therefore be beneficial to vaccinate isolated flocks known to be at risk, and/or to treat infected sheep with antibodies that bind to prion protein. The antibodies and/or peptides of the invention may similarly be used to clear all or part of the UK herd of BSE (cattle).

A further aspect of the invention lies in the use of the antibodies and/or peptides to treat humans presenting with the signs of, or identified as at risk of, CJD or new variant CJD.

In another embodiment, antibodies and/or peptides of the invention allow early diagnosis of a TSE infection based on tissue removed from, for example, the appendix, tonsils or through a lumbar puncture. With early diagnosis, the prospects of success in treatment, or at least prolonging life by treatment, are increased.

In yet another embodiment, antibodies and/or peptides of the invention are used to provide a wide-spread vaccination program in animals. It is envisaged that such a vaccination program could be carried out without prior diagnosis or testing.

The antibodies of the invention are optionally monoclonal antibodies that bind to prion proteins, preferably specifically to prion dimers, and can be used to treat prion disease.

As set out in more detail in the examples, prion strain 301V, a mouse passaged isolate, derived from a Holstein-Fresian cow terminally ill with BSE is used as an example of a prion strain. Infection is known to be produced by intracerebral inoculation and the incubation period required for the onset of clinical symptoms is remarkably uniform. That is to say, providing that the dose of infectious agent is sufficient then the classic signs of disease will appear at a defined time post-inoculation (in VM mice this is 120 days). For obvious reasons no-one has tested these properties on humans, however, the mouse bioassay is regarded as the closest available model and therefore a good indicator of BSE infection in man.

The methods and compositions of specific embodiments of the inventions are described in more detail below and are illustrated by the accompanying drawings in which:—

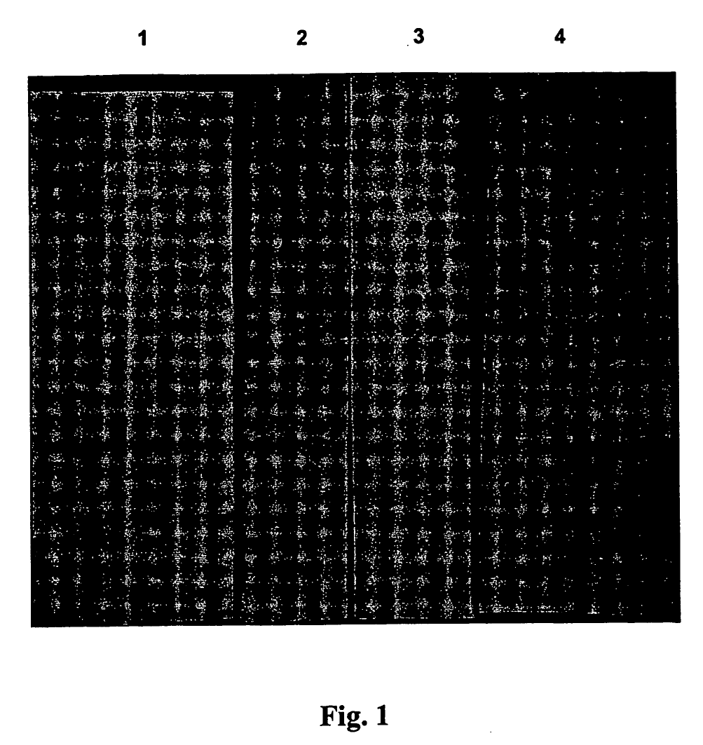

FIGS. 1 to 4 show blots of BSE (301V)-infected mouse brain homogenate, to illustrate binding of antibodies of the invention to prion dimer.

FIG. 5 shows the multiple alignment of selected mammalian and avian PrP^{c} proteins.

FIG. 6 shows the pair sequence distances of selected mammalian and avian PrP^{c} protein sequences.

### EXAMPLES

### Dimer Detection in Digested Mouse Brain

BSE (301V)-infected mouse brain homogenate was digested at neutral pH and 60° C. for 30 minutes with protease. Total protein digests were run on SDS-PAGE and transferred by Western blotting to nitro-cellulose membranes. These were cut into strips and probed with CAMR anti-prion antibodies (produced in rabbits). A second generic antibody (goat anti-rabbit) was conjugated to horseradish peroxidase and used with detection by TMB colorimetric substrate.

At the time, the expected result was that the results were the same as the control blot (number 7) using the anti-prion antibody mAb 6H4 (from Prionics, Switzerland). In this control blot, there is seen the typical three-banded pattern (glycosylation states) for protease-digested infectious-conformation prion protein (PrPSc).

However, the blots in this example did not show this pattern. Blot 1 uses a polyclonal antibody raised against a PPD-conjugated peptide corresponding to an N-terminal region of the prion molecule. Nothing is seen in the lanes. This section of the protein is susceptible to proteolysis, so it is not surprising to see nothing in the lanes (2 & 3)—see FIG. 1, blot 1 on left hand side. Lane 1 is a molecular weight marker.

Blot 2 has a second antibody raised against a peptide sequence further into the prion molecule. This shows at least 9 bands of varying intensity, approximately equidistant, at a molecular weight corresponding to a prion dimer with a range of glycosylation states—see blot 2 on FIG. 1.

Blot 3 antibody shows similar profile; blot 4 is also shown but its results are too poor quality to draw any conclusions—see blots 3 and 4 on FIG. 1.

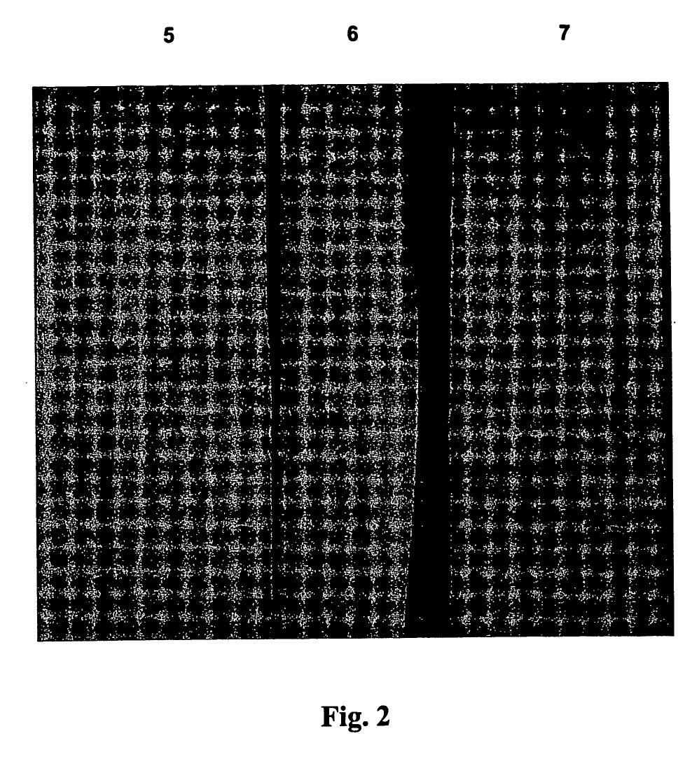

Blots 5 and 6, shown on FIG. 2 with the control blot 7, again show the multi-banded pattern.

Dimer Detection in Digested Mouse Brain

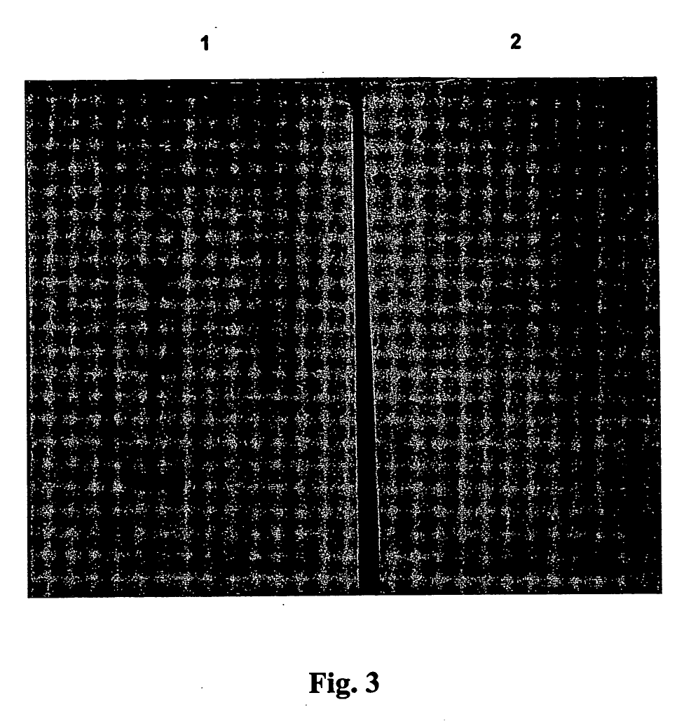

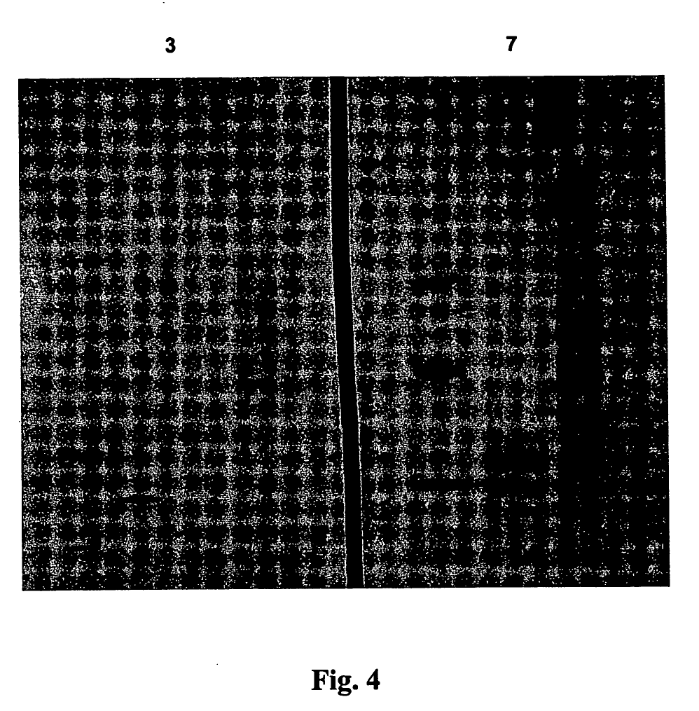

The above example was repeated, and the results shown in FIGS. 3 and 4.

Blot 1 shows molecular weight markers in lanes 1 and 5. Lane 2 is recombinant murine PrP showing recombinant murine PrP oligomers. Lane 3 shows lack of antibody response to protease-digested infectious mouse brain homogenate. Lane 4 is the antibody response in the undigested control.

Blot 2 is as above but shows the previous banding pattern in the protease digested sample.

Blot 3 shows the antibody 3 response. Here there is some response to recombinant murine PrP (lane 2). Lane 3 shows not only the multiple (dimeric PrP) banding pattern, but also some monomeric PrP response.

Blot 7 is the 6H4 mAb antibody control. Here there is good detection of recombinant murine PrP oligomers (lane 2). Lane 3 shows the heavily diglycosylated form of limitedly protease-treated PrPSc, plus the more minor monoglycosylated and non-glycosylated forms typical of BSE (301V) strain. No dimer detection is apparent.

Preparation of Antibodies Including Dimer Preferential Antibody

In the examples, we have used 6 polyclonal antibodies. Of these, three detect the dimer alone and do not bind the monomer, whereas one cross-reacts with both the monomer and the dimer.

The polyclonal sera were produced by immunisation of rabbits with synthesised prion mimetic peptides. These peptides were designed based on regions of high homology between human, mouse and bovine prion protein amino acid sequences.

SEQ ID No:s 4, 5, 7 and 8 produced the dimer-reactive antibodies.

The peptides were synthesised with a cysteine at both ends (see above) and with a cysteine at one end only. This method was used in order to present both the linear form and a loop structure of the antigen on the surface of the carrier protein.

The peptides were synthesised commercially and coupled to the carrier protein PPD (purified protein derivative), derived from an attenuated strain of the bacterium Mycobacterium bovis, which is lyophilised and used to conjugate to the peptide via a linker.

Anti-prion polyclonal antibodies were produced as follows:

A sample of pre-immune sera (−1 ml) was collected from each of a group of Dutch rabbits. The rabbits were injected with reconstituted freeze-dried Bacillus Calmette-Guerin (BCG) vaccine for intradermal use. A dose of 0.1 ml of reconstituted BCG vaccine was given in two sites in the scruff of the neck of the rabbit. After 4 weeks, 0.6 mg of each peptide-PPD conjugate was measured (0.3 mg of each of the 1 cysteine and 2 cysteine versions) and dissolved in 1 ml of sterile 0.9% saline.

An equal volume of incomplete Freunds adjuvant was added and 0.75 ml aliquots of the resulting emulsion were injected intra-muscularly into each hind limb, and 0.25 ml aliquots into two sites in the scruff of the neck per rabbit. After 4 weeks a boost injection was given comprising of the peptide-PPD conjugates prepared as in step 3 and 4. The boost injections consist of four 0.25 ml injections into the scruff of the neck of each rabbit. 7-14 days after the first boost injections, 4 ml test bleeds were taken, the sera was assessed by ELISA for antibody titre. A second boost injection was given 4-6 weeks after the first.

A third boost injection given 4-6 weeks later. A 4 ml test bleed was taken 6-8 weeks after the third boost injection and antibody titres determined by ELISA. A fourth boost injection was then given.

A 4 ml test bleed was taken 7-14 days after the fourth boost injection and antibody titre determined by ELISA. Terminal exsanguination was carried out and blood collected. The serum was separated by centrifugation and stored at minus 20° C.

Analysis of antibody titre was achieved using ELISA. The immunoassay plate was coated with the same peptides conjugated to a different carrier protein (KLH) in order to differentiate the response to the peptide from the response to the carrier protein.

Three of the antibodies produced by immunisation of the synthetic peptide sequences described bind preferentially to the dimer form of the molecule.

Immunisation Protocol Using PPD Coupled Prion-Derived Peptides for the Treatment of Prion Infection in Mice.

VM mice were inoculated intra-cerebrally with 20 ml of a 0.1% (w/v) suspension of BSE (301 V) infective mouse brain homogenate. After two weeks the mice were inoculated with BCG to prime the immune response to inoculated peptide conjugates. After a further 2 weeks the mice were inoculated with 100 ml of PPD-peptide conjugate mixed 1:1 with incomplete Freund's adjuvant or suitable controls. The peptides of the conjugates were selected from SEQ ID No:s 1-8, and corresponded as follows:—

Peptide 1 = SEQ ID No: 5
Peptide 2 = SEQ ID No 6
Peptide 3 = SEQ ID No 8

Three further inoculations at the same concentration of conjugate were administered at 2-weekly intervals. Mice were observed for clinical signs of the progression of prion disease over the next 2-6 months. Results of the experiment after 138 days are summarised in Table 2.

TABLE 2
Use of PPD coupled prion-derived peptides for the treatment of
prion infection in mice.

| | No. of | First Mouse | Last Mouse | Mean | SD |
|---|---|---|---|---|---|
| Treatment | Mice | culled on day:- | culled on day:- | (days) | (days) |
| BCG only | 9 | 123 | 133 | 129.7 | 5.0 |
| BCG + | 10 | 128 | 128 | 128.0 | 0.0 |
| PPD only | | | | | |
| BCG + | 10 | 133 | 133 | 133.0 | 0.0 |
| PPD- | | | | | |
| peptide 1 | | | | | |
| BCG + | 9 | 133 | 138 | 135.4 | 2.0 |
| PPD- | | | (2 mice still | | |
| peptide 2 | | | alive on day | | |
| | | | 138) | | |
| BCG + | 10 | 128 | 132 | 131.2 | 1.2 |
| PPD- | | | | | |
| peptide 3 | | | | | |

The results of this experiment indicate that immunisation with each of these PPD-peptides was successful in increasing the mean survival time of mice pre-exposed to TSE. Immunisation with PPD-peptide 1 and PPD-peptide 2 provided particularly notable increases in the mean survival time of mice preexposed to TSE. A statistical analysis of the results for these two peptides at the end of 138 days is provided below.

In interpreting the results of the above experiment, it should be recognised that the experiment represents an extremely rigorous test of the effectiveness of the peptides for the treatment of prion infection in mice, as the mice have been pre-exposed to TSE. Peptides of the invention thus are shown to increase survival time of mice pre-exposed to TSE and are suitable candidates for use in curative therapies.

The peptides are also suitable for use in prophylactic therapy, i.e. by exposing the animals to a peptide-carrier conjugate before exposure to the infectious agent.

Statistical Analysis of the Effect of PPD-Peptide 1 and PPD-Peptide 2 on the Mean Survival Time of Mice Pre-Exposed to TSE

One-way ANOVA: Group 2 (PPD-peptide 2) versus Group 8 (control-BCG only)

Analysis of Variance for Group 4

| Source | DF | SS | MS | F | P |
|---|---|---|---|---|---|
| Group 8 | 1 | 26.889 | 26.889 | 35.29 | 0.001 |
| Error | 7 | 5.333 | 0.762 | | |
| Total | 8 | 32.222 | | | |

| | | | | Individual 95% CIs For Mean | |
|---|---|---|---|---|---|
| | | | | Based on Pooled StDev | |
| Level | N | Mean | StDev | -------+---------+---------+--------- | |
| 123 | 3 | 133.000 | 0.000 | (------*-------) | |
| 133 | 6 | 136.667 | 1.033 | | (----*----) |

| | -------+---------+---------+--------- | | |
|---|---|---|---|
| Pooled StDev = 0.873 | 132.8 | 134.4 | 136.0 |

Two-Sample T-Test and Cl: Group 8 (control-BCG only), Group 2 (PPD-peptide 2)

Two-sample T for Group 8 vs Group 2

| | N | Mean | StDev | SE Mean |
|---|---|---|---|---|
| Group 8 | 9 | 129.67 | 5.00 | 1.7 |
| Group 2 | 9 | 135.44 | 2.01 | 0.67 |

Difference = mu Group 8 − mu Group 2
Estimate for difference: −5.78
95% CI for difference: (−9.78, −1.78)
T-Test of difference = 0 (vs not =): T-Value = −3.22
P-Value = 0.009 DF = 10

Mann-Whitney Test and Cl: Group 8 (control-BCG only), Group 2 (PPD-peptide 2)

| | | | | |
|---|---|---|---|---|
| Group 8 | N = | 9 | Median = | 133.00 |
| Group 2 | N = | 9 | Median = | 136.00 |

| | |
|---|---|
| Point estimate for ETA1-ETA2 is | −3.00 |

99.2 Percent CI for ETA1-ETA2 is (−13.00, −0.00)
W = 54.0
Test of ETA1 = ETA2 vs ETA1 < ETA2 is significant at 0.0031
The test is significant at 0.0016 (adjusted for ties)

Statistically, the observed difference between the control group and the group immunised with PPD-peptide 2 has been shown to be strongly significant.

Mann-Whitney Test and CI: Group 8 (control-BCG only), Group I (PPD-peptide 1)

| | | | | |
|---|---|---|---|---|
| Group 8 | N = | 9 | Median = | 133.00 |
| Group 1 | N = | 10 | Median = | 133.00 |

| | |
|---|---|
| Point estimate for ETA1-ETA2 is | −0.00 |

95.5 Percent CI for ETA1-ETA2 is (−10.00, 0.00)
W = 75.0
Test of ETA1 = ETA2 vs ETA1 < ETA2 is significant at 0.1182
The test is significant at 0.0628 (adjusted for ties)
Cannot reject at alpha = 0.05

Two-Sample T-Test and Cl: Group 8 (control-BCG only), Group I (PPD-peptide 1)

Two-sample T for control vs PPD-peptide 1

| | N | Mean | StDev | SE Mean |
|---|---|---|---|---|
| Group 8 | 9 | 129.67 | 5.00 | 1.7 |
| Group 1 | 10 | 133.000 | 0.471 | 0.15 |

Difference = mu Group 8 − mu Group 1
Estimate for difference: −3.33
95% CI for difference: (−7.19, 0.53)
T-Test of difference = 0 (vs not =): T-Value = −1.99
P-Value = 0.082 DF = 8

Statistically, the observed difference between the control group and the group immunised with PPD-peptide 1 is significant.

Immunisation of Human Subjects Infected with Prion Disease

Peptides are conjugated to PPD or BCG carrier proteins essentially as outlined above. The conjugate is formulated in such a way as to make it suitable for immunisation into clinical patients using methods and compositions known to those familiar with the art.

An immunisation protocol based on an initial immunisation with conjugate followed by two 2-weekly injections at 50% concentration followed by an additional injections at 2, 3 and 4 months is suitable for therapeutic treatment of individuals.

Production of Rabbit Polyclonal Antibodies and Mouse Monoclonal Antibodies

The production of polyclonal antibodies in rabbits is outlined below:

Dutch rabbits (approximately 2.5 kg) were allowed to settle in new accommodation for approximately two weeks and a sample of pre-immune serum (˜1 ml) collected from each. Inject each rabbit with reconstituted freeze-dried Bacillus Calmette-Guerin (BCG) vaccine for intradermal use (Statens Seruminsitut, Denmark). Dose with 0.1 ml of reconstituted BCG vaccine in each of two sites in the scruff of the neck of the rabbit. Leave all rabbits for 4-6 weeks before first injection of peptide-PPD conjugates.
Combine each of the peptide-PPD conjugates (1 ml) supplied with an equal volume of incomplete Freund's adjuvant and inject 0.75 ml aliquots of each resulting emulsion intramuscularly into each hind limb and 0.25 ml aliquots into two sites in the scruff of the neck per rabbit. Leave for 4-6 weeks.
Give first boost injection 4-6 weeks following first peptide-PPD conjugate incomplete Freund's adjuvant inoculation. Boost injections to comprise only four 0.25 ml injections into the scruff of each rabbit. Second boost to take place in 4-6 weeks time.
Take test bleed (−4 ml) 7-14 days following first boost injection.
Give second boost injection 4-6 weeks following first boost injection. Boost injection again to comprise only four 0.25 ml injections into the scruff of each rabbit. Third boost to take place in 4-6 weeks time.
Give third boost injection 4-6 weeks following second boost injection. Boost injection again to comprise only four 0.25 ml injections into the scruff of each rabbit.
Take second test bleed (−4 ml) 6-7 weeks following third boost injection and give fourth boost injection in approximately a further weeks time.
Take third test bleed (−4 ml) 7-14 days following fourth boost injection. Subject to serum analysis results, carry out terminal exsanguination within the next three days.

The immunisation protocol for the production of antibodies in mice is similar to that described above:—
1. Injection with reconstituted freeze dried Bacillus Calmette Guerin (BCG) Intradermal (day 0) 25 ml per mouse
2. Peptide—PPD conjugate in Freunds Incomplete Adjuvant ip (day 28) 80 mg/mouse
3. Repeat as DAY 28 (day 42) 40 mg/mouse
4. Repeat as DAY 28 (day 49) 40 mg/mouse
5. Tail vein test bleed (day 56)
6. Peptide—PPD conjugate (must be without adjuvant) iv (day 63)
7. Fusion on selected mice (day 66)

The immune response is assessed by ELISA using either plates coated with free peptide or peptide-KLH conjugate using doubling dilution to estimate end-point. Titres of between 1:51,200 and 1:102,000 are typically obtained for the immunised animals on ELISA plates coated with free peptide.

For the generation of monoclonal antibody cell lines the spleen of the immunised mouse is removed and fused with myeloma cell lines using standard methods (Antibodies; A laboratory Manual, Ed Harlow and David Lane, Chapter 6, p196-224. (Cold Spring Harbour Laboratory, 1988)). An example of a suitable method is provided below, although those familiar with the art will recognise that other similar protocols could be used for generation of antibody producing cell lines.

Preparing Splenocytes for Fusions

1. Sacrifice mouse. Aseptically remove spleen and place on a tissue culture dish containing 10 ml of serum free medium. Trim off and discard contaminating tissue from spleen.
2. Tease apart the spleen using 19 gauge needles on 1 ml syringes. Continue to tease until most of the cells have been released and the spleen has been tom into fine parts. Disrupt any cell clumps by pipetting. Transfer the cells and medium into sterile centrifuge tube.
3. Wash the tissue culture plate and tissue clumps with 10 ml serum free medium (prewarmed to 37° C.) and combine with the first 10 ml in the tube.
4. Allow the cell suspension to sit for approx. 2 min. Carefully remove the supernatant from the sediment and transfer to a fresh centrifuge tube.
   Fusion Technique

Prior to the fusion the myeloma cells that will serve as fusion partners must be removed from frozen stocks and grown.
1. Melt a vial of 0.3 g of PEG in a 50° C. water bath. Add 0.7 ml of medium without serum and transfer to a 37° C. water bath.
2. Centrifuge the spleen cells from the immunised animal at 400 g for 5 minutes. Simultaneously centrifuge 20 ml of myeloma cells. Resuspend both cell pellets in 5 ml of medium without serum.
3. Combine the two cell suspensions and transfer to a 15 ml round-bottomed centrifuge tube. Centrifuge for 5 min at 400 g. Carefully remove all medium.
4. Add 0.2 ml of PEG solution. Suspend the cells by lightly tapping the tube.
5. Centrifuge for 5 min at 400 g. Add 5 ml of medium without serum to disperse the pellet. Flick the tube, if necessary, to resuspend the cells. Do not pipet the cells. Then add 5 ml of medium with 20% Foetal calf serum.
6. Centrifuge for 5 min at 400 g. Remove the supernatant and resuspend the cells in 10 ml of medium supplemented with 20% foetal calf serum, 1×OPI and 1×HAT. Add the cells to 200 ml of medium supplemented with 20% foetal calf serum and 1×HAT.
7. Dispense 100 ml of cells into the wells of microtitre plates. Place at 37° C. in a CO₂ incubator.
8. Cells are fed by the addition of 100 ml of fresh medium supplemented with 20% Foetal calf serum containing HAT after 4-5 days.

OPI is a media supplement used to help support the growth of cells plated at low cell densities. It is a solution of oxaloacetate, pyruvate and insulin.

HAT—hypoxanthine, aminopterin and thymidine (drug selection media). Other commonly used drug selection methods are AH (azaserine and hypoxanthine).

Single Cell Cloning

After a positive tissue culture supernatant has been identified, the next step is to clone the antibody producing cell.
Using a multiwell pipettor, add 50 ml of medium with 20% FCS and 2×OPI to each well of a 96 well plate.
Remove 100 ml of hybridoma cell suspension and transfer to top left-hand well. Mix by pipetting.
Do doubling dilutions down the left-hand column of the plate. (8 wells, 7 dilution steps). Discard tip.
Do doubling dilutions across the plate using a 8-well multichannel pipette. Clones should be visible after a few days and ready to screen after 7-10 days. Select the best wells, grow up and repeat the cloning procedure.
   Generation of Humanized Anti-Prion Dimer Antibodies by CDR Grafting

The therapeutic potential of monoclonal antibodies that recognise prion-dimers may be significantly advanced by grafting the variable regions of the mouse antibodies onto the human variable domain by a process called CDR grafting (as described in Antibody engineering; a practical approach Eds McCafferty, J., Hoogenboom, H. R. and Chiswell, D. J. Oxford Univeristy Press 1996 and references therein). This method reduces the immunogenicity of the therapeutic antibody preparation by replacing much of the mouse antibody with the equivalent human protein. The method is briefly outlined below:

cDNAs encoding mouse monoclonals, produced and characterised as described above, are amplified by PCR using specific oligonucleotide primers designed to the heavy and light chain variable regions. These variable regions are cloned onto the constant regions of human antibodies to generate a chimeric antibody using methods known to those familiar with the art (as outlined in references including Antibody engineering; a practical approach Eds McCafferty, J., Hoogenboom, H. R. and Chiswell, D. J. Oxford Univeristy Press 1996). These chimeric antibodies are expressed recombinantly in either mammalian cells (e.g. Chinese hamster ovary cells; CHO) or E. coli.

Recombinant Expression of Antibody Fragments in E. coli.

For recombinant expression in E. coli the variable region either amplified from the original mouse monoclonal antibody cell line or from the humanised chimeric antibody is expressed either as an scFV fragment or a Fab fragment expressed into the cytoplasm of suitable E. coli strains (eg trxB mutants) or periplasmically using methods known to those familiar with the art and as outlined in Antibody engineering; a practical approach Eds McCafferty, J., Hoogenboom, H. R. and Chiswell, D. J. Oxford Univeristy Press 1996 and subsequent references.

To facilitate the purification of scFV fragments the variable regions are cloned into an expression vector containing a 6-histidine tag at either the N or C terminus. The addition of the tag has no effect on the recognition of the prion-dimer by the scFV or on function. For expression of the protein the clone in a trxB mutant E. coli strain (e.g. AD494 or BL21trxB; Novagen) is inoculated into 100 ml of LB with suitable antibiotic and grown overnight at 30° C. The culture is diluted 1:50 into fresh media and grown at 30° C. to an OD₆₀₀ₙₘ of 0.6-1. The culture is induced by addition of IPTG or other inducer appropriate to the expression system and the culture grown at 25° C. for a further 34 hours. Cell material is isolated by centrifugation.

Purification of Antibodies.

Antibody fragments are purified from either hybridoma supernatant, from CHO cells transfected with humanised IgG or from recombinant E. coli cultures using standard methods known to those familiar with the art. In brief, monoclonal antibodies are purified by affinity chromatography on Protein G or Protein A—sepharose columns following an (optional) ammonium sulphate precipitation from crude culture supernatant. Typically the crude supernatant from a hybridoma or CHO cell line is ammonium sulphate precipitated by addition of ammonium sulphate to 40% saturation and incubated for at least 1 hour at 4° C. The precipitate is collected by centrifugation at 15000 g for 30 min at 4° C. The pellet is resuspended in phosphate buffered saline (PBS) at a final concentration of approximately 2 mg/ml and loaded onto a protein-G-sepharose or protein-A sepharose column (Pharmacia) equilibrated in phosphate buffered saline according to manufacturers instructions. Purified antibody is eluted using 100 mM glycine pH 2.8 and collected directly into a concentrated phosphate buffer at high pH. The purified protein is dialysed extensively against PBS.

Expression and Purification of Recombinant Antibody Fragments in E. coli.

Purification of the recombinant scFV fragment from E. coli is carried out using standard methods. In the case of the his-tagged scFv fragment outlined in example 5 the cell pellet from the expression culture is lysed using either sonication or by proprietary detergent lysis (e.g. Bugbuster; Novagen) and clarified by centrifugation. The supernatant fraction is applied to an immobilised metal ion affinity chelate (IMAC) column (Pharmacia) loaded with Cu or Ni in 50 mM HEPES 150 mM NaCl pH7.4 or similar buffer. Following washing to remove non-specifically bound proteins the scFV is eluted using a gradient of 0-500 mM imidazole in the same buffer. The imidazole is removed by dialysis prior to storage. Other standard protein purification methods are also suitable for the isolation of recombinant antibody fragments from E. coli.

Formulation of Therapeutic Antibody Preparation for the Treatment of Prion Disease

Purified monoclonal antibodies or recombinant chimeric antibodies and fragments thereof are prepared as described above and shown to be free of endotoxin contamination. Antibody is formulated either with or without suitable carrier proteins (e.g. serum albumin), with or without 0.9% sodium chloride and in the presence or absence of suitable stabilising agents such as dextrose, sorbitol, sucrose or manitol. The antibody should be formulated at a concentration of 10 mg/ml.

Treatment of Prion Disease with Therapeutic Antibodies

For therapeutic application of the anti-prion dimer monoclonal antibody similar protocols can be used to those used for the treatment of lymphoma or other cancers with monoclonal antibodies. An example of a suitable dosing schedule is to prepare the formulated antibody as an infusion at a concentration of 1 mg/ml (final concentration) in 0.9% sodium chloride with 5% dextrose in water. The infusion is applied as a slow i.v. infusion over several hours. The concentration of antibody to be used is up to 400 mg/m² of body surface area and the dosage is repeated once weekly over eight weeks. Alternative dosing schedules such as 600 mg/m² once weekly for 4 weeks or other schedules known to those familiar with the art would also be suitable for administration of the antibody.

Improving the Uptake of Therapeutic Antibody Fragments Across the Blood-Brain Barrier

The effectiveness of the therapeutic application of anti-prion dimer antibodies depends on the ability of the antibody to reach the central nervous system (CNS). A number of the available methods to promote antibody access to the CNS, when used in conjunction with the inoculation of therapeutic antibody, may provide enhanced effectiveness over inoculation alone. Specific examples of such processes would be; 1) the conjugation of the therapeutic antibody to an anti-transferrin receptor antibody using standard methods outlined in: Lee H J. Engelhardt B. Lesley J. Bickel U. Pardridge W M. (2000) “Targeting rat anti-mouse transferrin receptor monoclonal antibodies through blood-brain barrier in mouse J Pharmacol Exp Ther. 292: 1048-52 and similar papers 2) the use of the drug Cereport (Alkermes Cambridge, Mass.) to lower the blood brain barrier following inoculation 3) conjugation of the antibody to an agent such as tetanus toxin receptor binding domain (Hc) for delivery via retrograde transport, 4) direct inoculation of recombinant antibody producing cells (such as glial, or Schwan cells) into the CNS.

The invention thus provides treatment of TSE infection and antibodies therefor.

1-41. (canceled)

42. A method of treatment of TSE infection, comprising administering an antibody that binds to a prion, wherein the prion comprises a PrP^{Sc} prion dimer that is infectious in animals.

43. A method according to claim 42, wherein the antibody is specific to PrP^{Sc} prion dimer.

44. A method according to claim 42, wherein the antibody is obtained by immunising an animal with a prion protein or an analogue thereof, or with a fragment of the protein or the analogue, obtaining an extract therefrom which contains antibodies, and isolating from said extract antibodies that bind to a prion, wherein the prion comprises a PrP^{Sc} prion dimer that is infectious in animals.

45. A method according to claim 42, wherein the antibody is obtained by immunising an animal with a peptide that comprises a fragment of prion protein.

46. A method according to claim 45, wherein the peptide is selected from SEQ ID NO:s 1 to 8, optionally supplemented by a cysteine residue at one or both ends.

47. A method according to claim 45, wherein the peptide is in a linear conformation, optionally a linear dimer, or a cyclic conformation, optionally a cyclic dimer.

48. A method according to claims 45, wherein the peptide comprises a repeated fragment of a prion protein.

49. A method according to claim 42, for treatment of a disease selected from the group consisting of Creutzfeld-Jacob disease; variant Creutzfeld-Jacob disease; Kuru; fatal familial insomnia; Gerstmann-Straussler-Scheinker syndrome; bovine spongiform encephalopathy; scrapie; feline spongiform encephalopathy; chronic wasting disease; and transmissible mink encephalopathy.

50. A method according to claim 42, wherein the antibody is a monoclonal antibody.

51. A pharmaceutical composition for treatment of TSE infection, comprising an antibody that binds to a prion, wherein the prion comprises a PrP^{Sc} prion dimer that is infectious in animals.

52. A composition according to claim 51, wherein the antibody is specific to PrP^{Sc} prion dimer.

53. A composition according to claim 51, wherein the antibody is obtained by immunising an animal with a prion dimer, obtaining an extract therefrom which contains antibodies, and isolating from said extract antibodies that bind a prion, wherein the prion comprises a PrP^{Sc} prion dimer that is infectious in animals.

54. A composition according to claim 51, wherein the antibody is obtained by immunising an animal with a peptide that comprises a fragment of prion protein.

55. A composition according to claim 54, wherein the peptide is selected from SEQ ID NO:s 1 to 8, optionally supplemented by a cysteine residue at one or both ends.

56. A composition according to claim 54, wherein the peptide is in a linear conformation, optionally a linear dimer.

57. A composition according to claim 54, wherein the peptide is in a cyclic conformation, optionally a cyclic dimer.

58. A composition according to claim 54, wherein the peptide comprises a repeated fragment of a prion protein.

59. A composition according to claim 51, for treatment of a disease selected from the group consisting of Creutzfeld-Jacob disease; variant Creutzfeld-Jacob disease; Kuru; fatal familial insomnia; Gerstmann-Straussler-Scheinker syndrome; bovine spongiform encephalopathy; scrapie; feline spongiform encephalopathy; chronic wasting disease; and transmissible mink encephalopathy.

60. A method of obtaining an antibody, comprising immunising an animal with an antigen, wherein the antigen comprises a peptide that comprises a fragment of a prion protein or of an analogue of a prion protein, obtaining antibodies from the animal and identifying antibodies that bind to prion, wherein the prion comprises a PrP^{Sc} prion dimer that is infectious in animals.

61. A method according to claim 60, wherein the antigen comprises a carrier covalently linked to the peptide, optionally via a linker.

62. A method according to claim 61, further comprising sensitising the animal to the carrier.

63. A method according to claim 62, wherein sensitising the animal to the carrier comprising administering a priming antigen that stimulates an immune response to the carrier.

64. A method according to claim 63, wherein the carrier comprises a heat shock protein.

65. A method according to claim 63, wherein the carrier is a Mycobacterial protein and the priming antigen is administered by administering Bacillus Calmette-Guerin vaccine.

66. A method according to claim 60, wherein the peptide is in a cyclic form.

67. A method according to claim 60, wherein the antigen comprises a composite of repeats of the peptide.

68. A method according to claim 67, wherein the antigen comprises a linear or cyclic dimer of the peptide.

69. An antigen comprising a fragment of at least 7 amino acids of a prion protein, wherein the antigen is capable of stimulating production of an antibody that binds to a prion comprising a PrP^{Sc} prion dimer that is infectious in animals.

70. An antigen according to claim 69, wherein the fragment is a cyclic fragment of the prion protein.

71. An antigen according to claim 69, wherein the fragment is a linear fragment of the prion protein.

72. An antigen according to claim 69, wherein the antigen comprises repeats of said fragment.

73. An antigen according to claim 72, wherein the antigen comprises a dimer of said fragment.

74. An antigen according to claim 69, wherein the fragment comprises any one of SEQ ID NO:s 1 to 8.

75. A conjugate, for stimulating production of an antibody, comprising a carrier linked to an antigen according to claim 69.

76. A method of immunizing an animal against TSE infection, comprising administering an antigen according to claim 69, to the animal.

77. A method according to claim 76, wherein the antigen is administered subsequently to or simultaneously with a priming antigen that stimulates a response to the antigen.


SEQUENCE LISTING (8 sequences)

SEQ ID NO: 1 - protein, 15 aa, Artificial Sequence
  Synthetic
CGGSRYPGQGSPGGC

SEQ ID NO: 2 - protein, 12 aa, Artificial Sequence
  Synthetic
CGGNRYPPQGGC

SEQ ID NO: 3 - protein, 16 aa, Artificial Sequence
  Synthetic
CKKRPKPGGGWNTGGC

SEQ ID NO: 4 - protein, 11 aa, Artificial Sequence
  Synthetic
CGGWGQPHGGC

SEQ ID NO: 5 - protein, 23 aa, Artificial Sequence
  Synthetic
CGGYMLGSAMSRPIIHFGNDYEC

SEQ ID NO: 6 - protein, 26 aa, Artificial Sequence
  Synthetic
CDYEDRYYRENMHRYPNQVYYRPVDC

SEQ ID NO: 7 - protein, 25 aa, Artificial Sequence
  Synthetic
CVNITIKQHTVTTTTKGENFTETDC

SEQ ID NO: 8 - protein, 19 aa, Artificial Sequence
  Synthetic
CITQYQRESQAYYQRGASC